(19) United States
(12) Patent Application Publication
(10) Pub. No.: US 2024/0239657 A1
(43) Pub. Date: Jul. 18, 2024

(54) METHOD OF PREPARATION AND PROPERTIES OF PURE HYPOCHLOROUS ACID, HOCL

(71) Applicant: WALKER CANCER RESEARCH INSTITUTE, INC., Boynton Beach, FL (US)

(72) Inventor: Eduardo PALOMINO, Troy, MI (US)

(73) Assignee: WALKER CANCER RESEARCH INSTITUTE, INC., Boynton Beach, FL (US)

(21) Appl. No.: 18/390,835

(22) Filed: Dec. 20, 2023

Related U.S. Application Data
(63) Continuation-in-part of application No. 17/394,680, filed on Aug. 5, 2021.

(51) Int. Cl. C01B 11/04

(52) U.S. Cl. CPC C01B 11/04; C01P 2002/82

(57) ABSTRACT

A method for preparing pure hypochlorous acid includes a step of combining a predetermined amount of sodium hypochlorite with a predetermined volume of a nonpolar solvent and a predetermined amount of a weak acid to form a biphasic mixture that includes an aqueous phase and an organic phase. The biphasic mixture is agitated and then the organic phase is separated from the aqueous phase. The nonpolar solvent is removed from the organic phase to obtain the pure hypochlorous acid.

### CROSS-REFERENCE TO RELATED APPLICATIONS

This application is a continuation-in-part of U.S. application Ser. No. 17/394,680 filed Aug. 5, 2021, the disclosure of which is hereby incorporated in its entirety by reference herein.

### TECHNICAL FIELD

In at least one aspect, the present invention is related to methods to produce pure hypochlorous acid (HOCl).

### BACKGROUND

Hypochlorous acid (HOCl), the acid precursor of bleach or sodium hypochlorite, is described to exist only as a 25% aqueous solution, greenish yellow in color, that may be stored for a few days at −20° C. However, this composition slowly decomposes to chlorine, oxygen gas, and perchloric acid (Merck Index 10th Ed. Pag. 4783). Its preparation, according to the same source, is achieved by the action of water on chlorine (Cady, Inorg. Syn. 5, 160, 1957); the entire disclosure of which in hereby incorporated by reference. The 25% solution described in the Merck Index was made by distillation at low pressure of chlorine hydrate and mercuric oxide as shown in the following equation:

$$2 {Cl}_{2} {\cdot 6H}_{2} O + HgO ⟶ 2 HClO + {HgCl}_{2} + 11 {H}_{2} O$$

Sodium hypochlorite (NaOCl or bleach) has been used extensively since it was discovered in 1789 by Berthollet. However, much of its chemistry is poorly understood. A Sodium Hypochlorite Handbook issued by Oxychem in 2014, gives an account of its composition at different pH value; the entire disclosure of which in hereby incorporated by reference. As disclosed in the Handbook, hypochlorous acid (HOCl) is the predominant compound at pH between 2 and 7.4, approaching 100% between pH 4 and 5.5.

The pH profile from A Sodium Hypochlorite Handbook suggests that pure HOCl can be produced by simple acidification of bleach and several vendors market such aqueous solution as hypochlorous acid. The federal government has established that hypochlorous acid, of CAS #7790-92-3, must not exceed 200 ppm of chlorine when used as a pesticide solution in agriculture. In notification FCN 2161 of July 2021, the FDA indicates that hypochlorous acid be electrolytically generated by the manufacturer. Many patents and applications have been issued in which an aqueous solution of an acidified sodium hypochlorite is produced in different degrees of purity or stabilized by salt or buffer. Some examples of those follow:

In US Pat. Appl. No. 2009/0258083, NaOCl (13%) is treated with HCl (33%) to a pH 2.8-4.0.

In U.S. Pat. No. 5,027,627, the NaOCl is produced first by reaction of sodium hydroxide with chlorine gas followed by pH adjustment.

In U.S. Pat. No. 4,190,638, NaOCl is produced using an electrolytic cell.

In US Pat. Appl. No. 2021/0238752, NaOCl is produced by electrolysis of NaCl

In U.S. Pat. No. 11,097,945, NaOCl is produced also by electrolysis of NaCl and the HOCl is produced by adjusting the pH to a range 3 to 8 by adding a weak acid, such as acetic acid, and a salt of the selected weak acid to produce a buffer. This patent recognizes that the strong acid HCl, used in most applications, can cause an increase in the production of chlorine gas, hence the reason for the use of a weak acid.

In an application for inclusion in the 2021 WHO Essential Medicines List entitled “Hypochlorous acid (HOCl) for disinfection, antiseptics, and wound care” the so-called Brio HOCl is produced electrochemically from aqueous NaCl solution followed by adjustment of pH of the NaOCl formed to a value less than 5.5.

The hypochlorous acid described in all the literature above corresponds to an aqueous mixture containing different salts, such as sodium chloride, sodium acetate, etc., derived from the manufacturing process, in addition to HOCl. None of those preparations contain pure HOCl or an aqueous solution of pure HOCl.

HOCl can be considered a derivative of water in which a hydrogen atom is substituted by the bulky chlorine atom as shown in a chemically accurate 3D representation in FIG. 1. The bulky chlorine atom makes the molecule less polar than water and suggests it has solubility in nonpolar solvents. Despite this fact, no patent or literature publication has used a nonpolar solvent for extracting hypochlorous acid, and no patent or literature publication describes the production of pure, undiluted hypochlorous acid or its physicochemical properties.

Accordingly, there is a need for new methods for forming hypochlorous acid.

### SUMMARY

In at least one aspect, a method for preparing pure hypochlorous acid is provided. The method includes a step of combining a predetermined amount of sodium hypochlorite with a predetermined volume of a nonpolar solvent and a predetermined amount of a weak acid to form a biphasic mixture that includes an aqueous phase and an organic phase. The biphasic mixture is agitated, and then the organic phase is separated from the aqueous phase. The nonpolar solvent is removed from the organic phase to obtain the pure hypochlorous acid.

In another aspect, a method for preparing pure hypochlorous acid is provided. The method includes a step of combining with agitation a predetermined amount of bleach with a predetermined volume of a nonpolar solvent and a predetermined amount of a weak acid, the bleach including sodium hypochlorite. The nonpolar solvent that includes the hypochlorous acid is separated from an aqueous phase. The nonpolar solvent is treated with a dehydrating agent to form dried hypochlorous acid. The nonpolar solvent containing the dried hypochlorous acid is decanted for collection, and the nonpolar solvent is removed by vacuum aspiration to obtain the pure hypochlorous acid.

In another aspect, a method for making pure hypochlorous acid is provided. The method includes a step involving the reaction of commercial sodium hypochlorite (bleach) with a weak acid, such as acetic acid; extracting the hypochlorous acid produced from the aqueous mixture by shaking it with a nonpolar solvent, such as ethyl acetate; removing the nonpolar solvent by vacuum evaporation after removing the excess water with a dehydrating agent such as molecular sieves (zeolites) or anhydrous sodium sulfate. The hypochlorous acid thus produced is a clear liquid with a faint chlorine smell, denser than water (d=1.45 g/ml); freezing point −10° C.; IR (NaCl plate) displays one absorption for the OH at 3437 cm⁻¹ and two absorptions (bending at 1642 cm⁻¹ and stretching at 1111 cm⁻¹) for the Cl; cyclic voltammetry (CV) gives a reducing maximum at 440 mV; the concentration and purity of the HOCl produced was measured by titration, using phenolphthalein as indicator, with standardized NaOH solution of the HCl produced from the reaction of HOCl with H2O2.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1. 3D rendering of H₂O and HOCl.

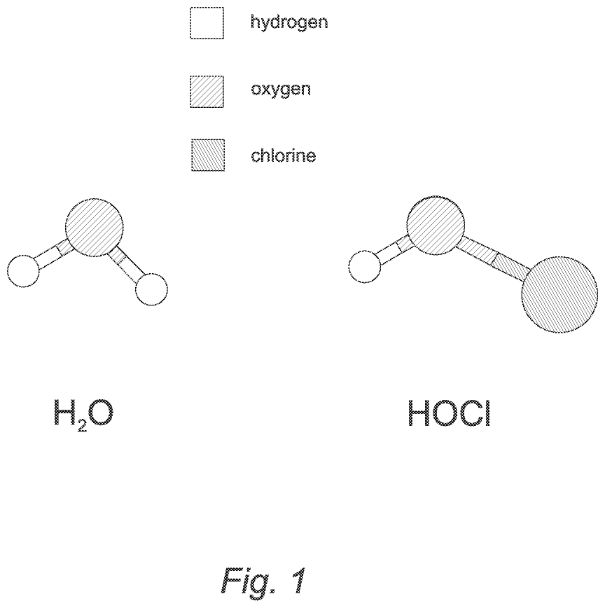

FIG. 2. IR chromatogram of pure hypochlorous acid (HOCl) taken on a sodium chloride plate taken on a Nicolet iS5 instrument.

FIG. 3. Cyclic Voltammogram of pure hypochlorous acid (HOCl) taken in 1M solution of potassium nitrate (KNO₃) using a platinum working electrode, platinum wire auxiliary electrode, and silver/silver chloride reference electrode on a Basi Epsilon EC instrument.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including” or “comprising.” Typically, this phrase is used to denote that an object is formed from a material.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entireties are hereby incorporated by reference into this application to more fully describe the state of the art to which this invention pertains.

The term “weak organic acid” refers to an acid having a pKₐ from 2 to 7.5.

The term “weak organic acid” refers to a carboxylic acid, dicarboxylic acid, or tricarboxylic acid having a pKₐ from 2 to 7.5. In a refinement, refers to a carboxylic acid, dicarboxylic acid, or tricarboxylic acid having a pKₐ of at least 2, 2.5, 3, 3.5, or 4 and of at most 7.5, 7, 6.5, 6, 5.5 or 5. Examples of weak organic acids include but are not limited to, acetic acid, formic acid, propionic acid, butyric acid, benzoic acid, lactic acid, and oxalic acid. It is noteworthy that acetic acid has a pKₐ of about 4.76.

The term “pure hypochlorous acid” means that the hypochlorous acid prepared from the methods below includes compounds other than hypochlorous acid in an amount of less than 1 weight percent. With respect to solutions, a pure hypochlorous acid solution includes compounds other than hypochlorous acid and the solvents in an amount of less than 1 weight percent.

In an embodiment, a method for preparing pure hypochlorous acid is provided. The method includes a step of combining a predetermined amount of sodium hypochlorite with a predetermined volume of a nonpolar solvent and a predetermined amount of a weak acid to form a biphasic mixture that includes an aqueous phase and an organic phase. The biphasic mixing is agitated to promote mixing of the reagents. The organic phase is separated from the aqueous phase. In a refinement, the separated organic phase is treated with a dehydrating agent. The nonpolar solvent is removed from the organic phase to obtain the pure hypochlorous acid. In a refinement, the nonpolar solvent is removed by vacuum aspiration.

In another aspect, the aqueous phase is extracted with one or more additional amounts of the nonpolar solvent that are subsequently recombined with the organic phase separated in step c).

In another aspect, the molar ratio of sodium hypochlorite to the weak acid is about 1:1. In some refinements, the molar ratio of sodium hypochlorite to the weak acid is at least 0.7:1, 0.8:1, 0.9:1, 1:1, 1.1:1, or 1.2:1. In other refinements, the molar ratio of sodium hypochlorite to the weak acid is at most 2:1, 1.5:1, 1.4:1, 1.3:1, 1.2:1, or 1.1:1.

In another aspect, the weak acid has a pKa from 3 to 5. In a refinement, the weak acid is a weak organic acid. An example of a particularly useful weak acid is acetic acid.

In another aspect, the nonpolar solvent has a dielectric constant that is less than or equal to 10. In a refinement, the nonpolar solvent is an aprotic solvent. An example of a particularly useful solvent is ethyl acetate.

In another aspect, the method further includes a step of weighing the pure hypochlorous acid and calculating its purity by titrating hydrochloric acid with sodium hydroxide where the hydrochloric acid is produced by the reaction of a hypochlorous acid with hydrogen peroxide.

In another aspect, sodium hypochlorite is obtained from bleach, which can include sodium hypochlorite in an amount from about 2 to 12 weight percent in water. Typically, bleach includes about 6 weight percent sodium hypochlorite in water.

Characteristically, the pure hypochlorous acid has the following physicochemical properties: clear liquid with a faint chlorine smell; density 1.45 g/ml; freezing point −10° C.; IR spectrum (NaCl plate): absorption at 3437 cm⁻¹ for the OH group and two absorptions (bending at 1642 cm⁻¹ and stretching at 1111 cm⁻¹) for the Cl atom; and cyclic voltammetry (CV) showing a reducing maximum at 440 mV.

The pure hypochlorous acid produced using the method of the present invention has several advantages to the impure mixtures that are qualified as hypochlorous acid in the market. First, is the weight of the acid mixture compared to the pure acid. For instance, a 100 g of 6% NaOCl (regular commercial bleach concentration) used to prepare 4.2% of the acid is equivalent to 4.2 g of the pure acid. This is a greater than 20 times reduction in weight or volume. Second, the purity of the acid of the present invention facilitates its use as a reagent in either aqueous or nonpolar solvent. Third, the acid of the present invention is qualified as reagent grade, so many reactions can be performed in many solvents and the results can be easily evaluated, products separated, and mechanisms of reactions studied.

The following examples illustrate the various embodiments of the present invention. Those skilled in the art will recognize many variations that are within the spirit of the present invention and scope of the claims.

The present invention provides a method for the preparation of pure hypochlorous acid via acidification with a weak acid of a solution of sodium hypochlorite (bleach) followed by extraction with a nonpolar (organic) solvent; removal of the organic solvent by an evaporative method and removing the excess water using a dehydrating agent. The pure hypochlorous acid produced using the above method is a clear colorless liquid, stable at room temperature for several days and stable indefinitely under refrigeration. It has a faint bleach odor. Its purity and/or concentration are evaluated by measuring the amount of HCl produced on its reaction with hydrogen peroxide (H₂O₂) using phenolphthalein as indicator, according to the following equations:

$$\begin{matrix}a ) & \begin{matrix}HOCl + {H}_{2} {O}_{2} & ⟶ & HCl + {O}_{2} + {H}_{2} O\end{matrix} \\ b ) & \begin{matrix}HCl + NaOH & ⟶ & NaCl + {H}_{2} O\end{matrix}\end{matrix}$$

Pure, undiluted hypochlorous acid (HOCl) has not been reported in the patent or scientific literature. What had been reported is a diluted aqueous solution of a mixture produced by acidifying mostly the sodium salt (sodium hypochlorite) with different acids, especially hydrochloric acid or acetic acid. Those mixtures contain variable amounts of salts including sodium chloride, sodium acetate, chlorine and hypochlorous acid.

The present invention makes use of the unreported solubility of hypochlorous acid in nonpolar solvent and uses inexpensive, commercially available sodium hypochlorite (bleach), eliminating the need for expensive and cumbersome electrolytic processes. Extracting hypochlorous acid with a nonpolar solvent produces pure hypochlorous acid free of ionic impurities such as sodium chloride, sodium acetate, and other salts present in the commercial bleach. Removing the water from the extracted hypochlorous acid using known dehydrating agents such as anhydrous sodium sulfate, Na₂SO₄, which absorbs 7 units of water to become Na₂SO₄.7H₂O, or zeolites that trap the water inside its pores, produces pure hypochlorous acid, HOCl.

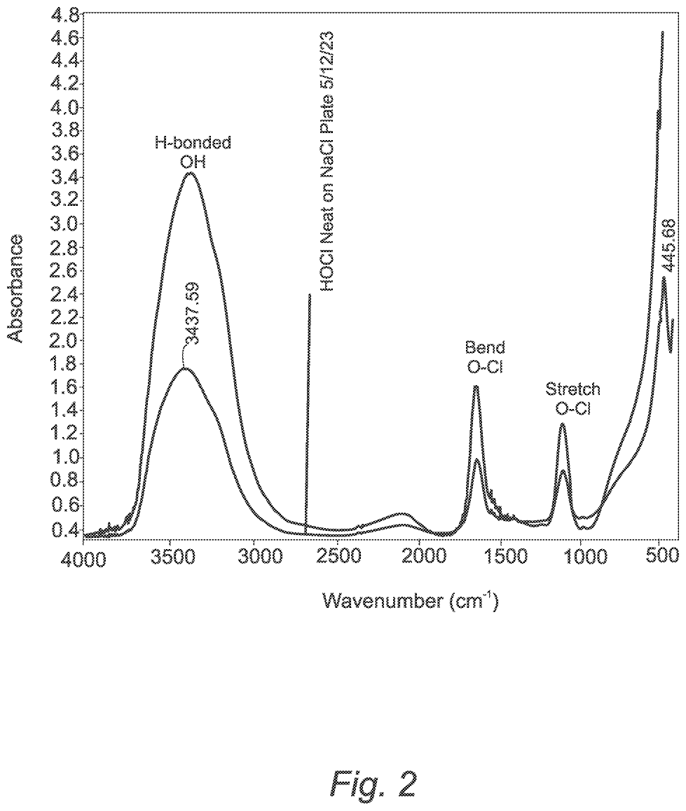

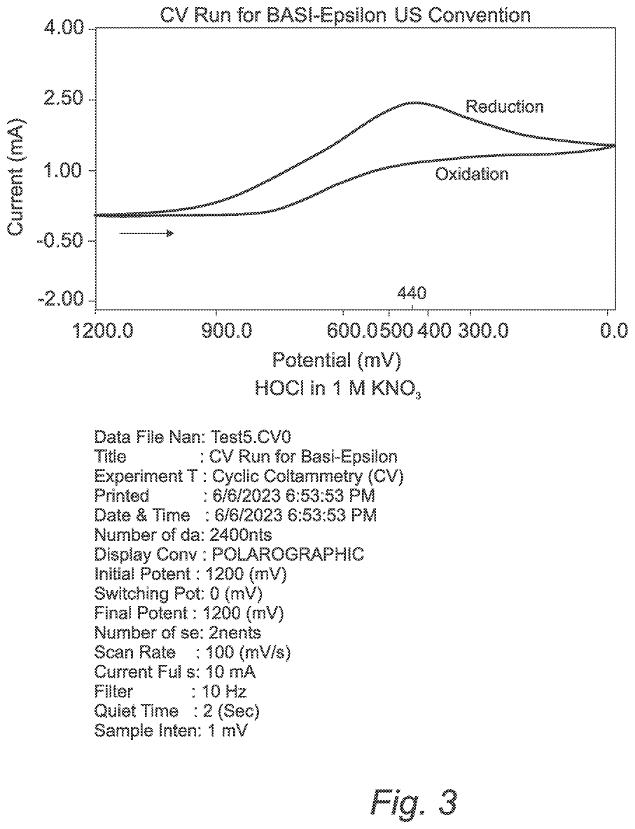

The hypochlorous acid extracted and dried is a clear liquid with a faint chlorine smell, denser than water (d=1.45 g/ml); freezing point −10° C.; IR (NaCl plate), FIG. 2, showing one absorption for the OH at 3437 cm⁻¹ and two absorptions (bending at 1642 cm⁻¹ and stretching at 1111 cm⁻¹) for the Cl; cyclic voltammetry (CV), FIG. 3, gives a reducing maximum at 440 m V. Evaluation of its purity is done by measuring the amount of hydrochloric acid produced by its reaction with hydrogen peroxide set forth above.

### Example 1

A solution of 100 ml of commercial bleach (Sodium hypochlorite, NaOCl, 6% in water) is placed in a 500 ml separatory funnel along with 300 ml of pure ethyl acetate. To this biphasic mixture 4.8 g of pure acetic acid is added, and the mixture shaken for 3 to 5 minutes. The bottom aqueous phase is separated and extracted again two times with 100 ml of ethyl acetate each time. The ethyl acetate fractions (500 ml in total) are combined, stirred 5 min with 2 g of solid sodium sulfate (Na₂SO₄), decanted and the ethyl acetate removed and collected by rotavapor concentration. The yield of pure hypochlorous acid (HOCl) was 4.1 g, equivalent to a complete stoichiometric conversion.

The purity of the hypochlorous acid produced above was evaluated by measuring the amount of hydrochloric acid produced from 1 g of HOCl reacting with hydrogen peroxide 50%, according to the reaction set forth above.

To 1 g of pure hypochlorous acid in a 100 ml beaker, 1.44 g of 50% hydrogen peroxide was added. When the evolution of oxygen had stopped, 10 ml of water and 2 drops of phenolphthalein were added, and the solution was titrated with a 1M solution of sodium hydroxide until the color of the solution changed from colorless to pink when 19 ml of sodium hydroxide had been added. That volume corresponded to 0.72 g of HCl which corresponded to 1 g of HOCl, indicating that the purity of hypochlorous acid is 100%.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for preparing pure hypochlorous acid comprising:
a) combining a predetermined amount of sodium hypochlorite with a predetermined volume of a nonpolar solvent and a predetermined amount of a weak acid to form a biphasic mixture that includes an aqueous phase and an organic phase;
b) agitating the biphasic mixture;
c) separating the organic phase from the aqueous phase; and
d) removing the nonpolar solvent from the organic phase to obtain the pure hypochlorous acid.

2. The method of claim 1, wherein the organic phase separated in step c) is treated with a dehydrating agent.

3. The method of claim 1, wherein the aqueous phase is extracted with one or more additional amounts of the nonpolar solvent that are subsequently recombined with the organic phase separated in step c).

4. The method of claim 1, wherein the nonpolar solvent in step d) is removed by vacuum aspiration.

5. The method of claim 1, wherein a molar ratio of sodium hypochlorite to the weak acid is about 1:1.

6. The method of claim 1, wherein the weak acid has a pKₐ from 3 to 5.

7. The method of claim 1, wherein the weak acid is a weak organic acid.

8. The method of claim 1, wherein the weak acid is acetic acid.

9. The method of claim 1, wherein the nonpolar solvent has a dielectric constant that is less than or equal to 10.

10. The method of claim 1, wherein the nonpolar solvent is ethyl acetate.

11. The method of claim 1 further comprising weighing the pure hypochlorous acid and calculating its purity by titrating hydrochloric acid with sodium hydroxide, wherein the hydrochloric acid is produced by reaction of a hypochlorous acid with hydrogen peroxide.

12. The method of claim 1, wherein the sodium hypochlorite is obtained from bleach.

13. The method of claim 1, wherein the pure hypochlorous acid is has the following physicochemical properties: clear liquid with a faint chlorine smell; density 1.45 g/ml; freezing point −10° C.; IR spectrum (NaCl plate): absorption at 3437 cm⁻¹ for the OH group and two absorptions (bending at 1642 cm⁻¹ and stretching at 1111 cm⁻¹) for the Cl atom; and cyclic voltammetry (CV) showing a reducing maximum at 440 mV.

14. A method for preparing pure hypochlorous acid comprising:
a) combining with agitation a predetermined amount of bleach with a predetermined volume of a nonpolar solvent and a predetermined amount of a weak acid, the bleach including sodium hypochlorite;
b) separating the nonpolar solvent that includes the hypochlorous acid;
c) treating the nonpolar solvent with a dehydrating agent to form dried hypochlorous acid;
d) decanting the nonpolar solvent containing the dried hypochlorous acid; and
e) removing the nonpolar solvent by vacuum aspiration to obtain the pure hypochlorous acid.

15. The method of claim 14, wherein a molar ratio of sodium hypochlorite to the weak acid is about 1:1.

16. The method of claim 14, wherein the weak acid has a pKₐ from 3 to 5.

17. The method of claim 14, wherein the weak acid is a weak organic acid.

18. The method of claim 14, wherein the weak acid is acetic acid.

19. The method of claim 14, wherein the nonpolar solvent has a dielectric constant that is less than or equal to 10.

20. The method of claim 14, wherein the nonpolar solvent is ethyl acetate.

21. The method of claim 14 further comprising weighing the pure hypochlorous acid and calculating its purity by titrating hydrochloric acid with sodium hydroxide, wherein the hydrochloric acid is produced by reaction of a hypochlorous acid with hydrogen peroxide.
